(19) United States
(12) Patent Application Publication
(10) Pub. No.: US 2014/0074006 A1
(43) Pub. Date: Mar. 13, 2014

(54) Maxillofacial bandage

(71) Applicant: Richard S. Manley, JR., Longview, TX (US)

(72) Inventor: Richard S. Manley, JR., Longview, TX (US)

(73) Assignee: Avcor Health Care Products, Inc., Arlington, TX (US)

(21) Appl. No.: 13/987,865

(22) Filed: Sep. 10, 2013

Related U.S. Application Data
(60) Provisional application No. 61/743,812, filed on Sep. 12, 2012.

(51) Int. Cl. A61F 13/12; A61F 7/02; A61F 13/00

(52) U.S. Cl. CPC A61F 13/122; A61F 13/00038; A61F 7/02
USPC 602/74

(57) ABSTRACT

Therapeutic bandages or support wrappings in the form of elongated strips of stretchable material carrying a pocket on one surface are used for therapeutic treatment and support of facial and jaw tissues. The pocket material is non-elastic and moisture-impervious. A thermal packet positioned in the pocket is held adjacent an affected body part and compressive force applied thereto by adjusting the tension on the elastic wrapping strip.

This invention relates to therapeutic bandages or support wrappings for therapeutic treatment and support of facial and jaw tissues. More particularly, it relates to therapeutic bandages for use in treatment of facial and jaw tissues during post-operative recovery or recovery from other insult or injury.

Facial and jaw tissue recovery from maxillofacial injury or surgery usually requires application of cold and/or compressive pressure to immobilize the tissue and reduce swelling during recovery and healing. Support bandages for such use have been devised which comprise a simple non-elastic wrapping applied to the affected area and held in place by clamps, fasteners or the like. Some such bandages include pockets for containing thermal packets such as cold gel paks or the like to be held adjacent affected body parts. Such prior art bandages, however, are limited in applicability and effectiveness since they are usually comprised of non-elastic materials which are non-permeable and non-absorbent. As a result, the compressive force applied by the bandage requires constant re-adjustment and the non-permeable material prevents air circulation, causing discomfort to the patient.

The present invention provides bandages adapted for maxillofacial application comprising an elastically expandable (stretchable) elongated strip of wrapping material defining a length, a width, opposite ends, an inner surface and an outer surface; at least one pocket formed of breathable but non-porous material attached to or formed on one surface of the strip which is adapted to contain a thermal packet; and closure means adapted to secure the strip in a wrapped position on a human body part and apply a relatively constant compressive pressure to said body part. Such bandages not only support a thermal packet adjacent the affected body part, they also apply a continuous predetermined compressive pressure to aid in immobilization of the affected tissue to reduce swelling and encourage healing. Other features and advantages of the invention will become more readily understood from the following detailed description taken in connection with the appended claims and attached drawing in which:

FIG. 1 is a plan view of the inner surface of a bandage employing the principles of the invention;

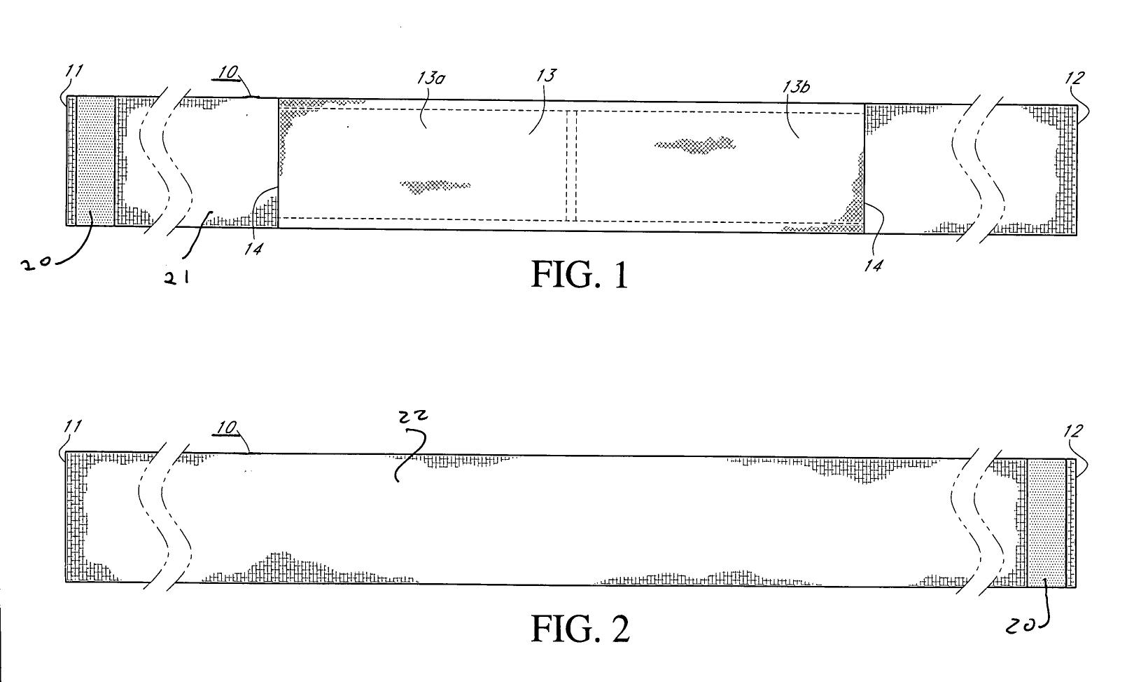

FIG. 2 is a plan view of the outer surface of the bandage of FIG. 1;

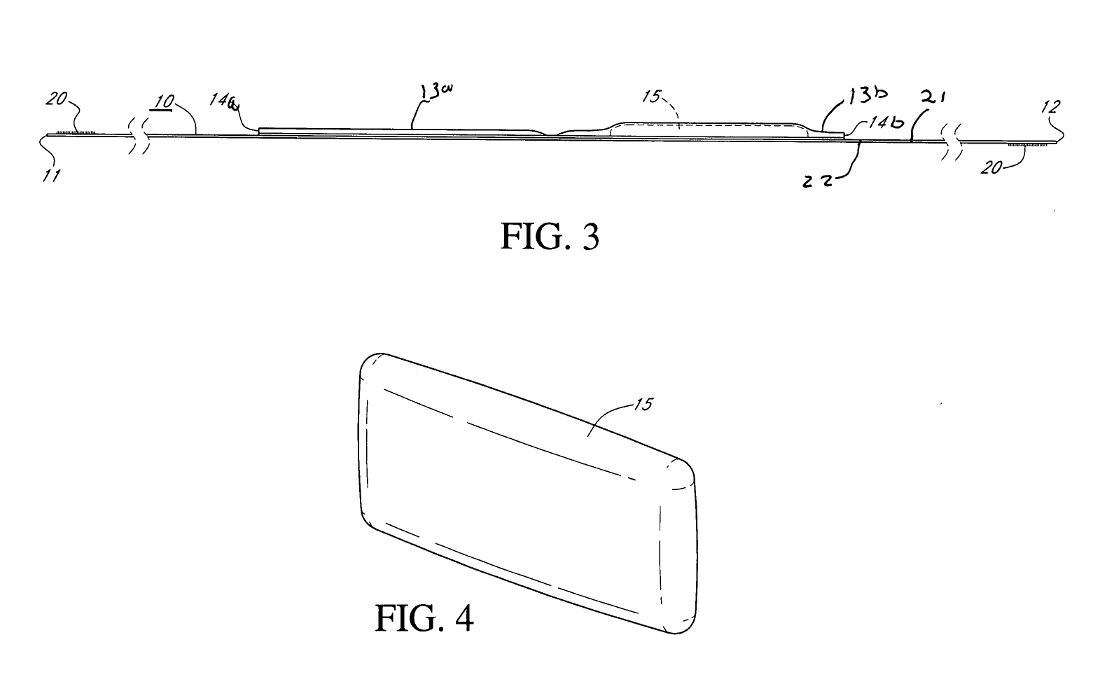

FIG. 3 is an edge view of the bandage of FIG. 1;

FIG. 4 is a perspective view of an exemplary gel pak for use in connection with the bandage of the invention;

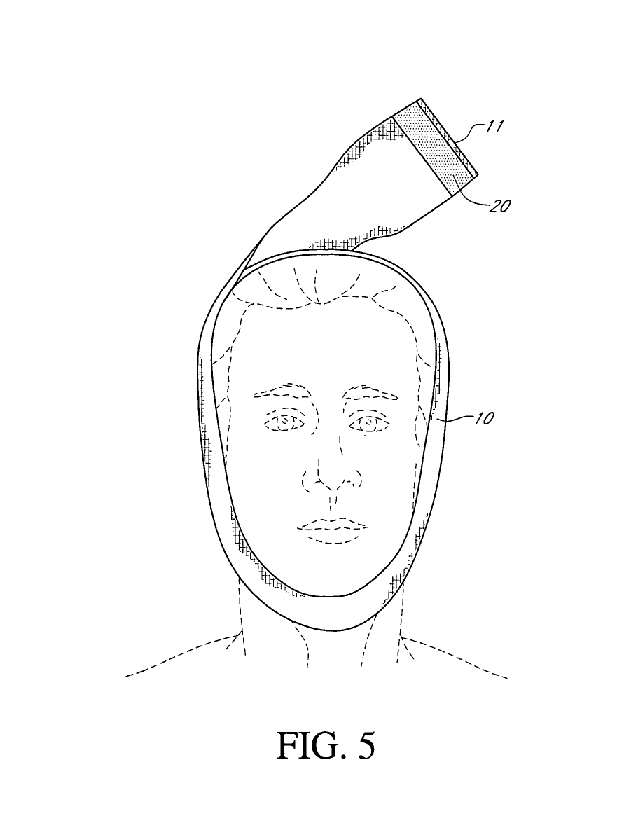

FIG. 5 is a demonstrative illustration of application of the bandage of the invention; and

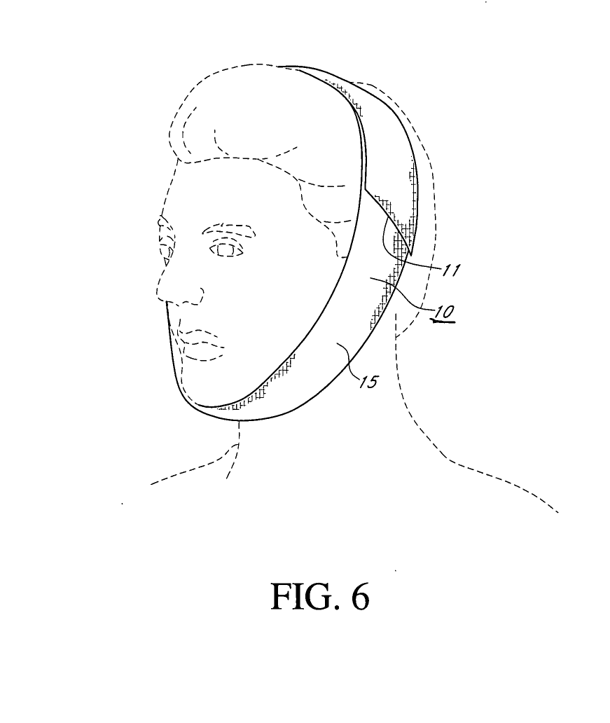

FIG. 6 is a demonstrative illustration of use of the bandage of the invention to apply compressive force to a thermal packet in treatment of a maxillofacial wound or injury.

The above-described drawing is incorporated into and forms part of the specification to illustrate exemplary embodiments of the invention. Throughout the drawing like reference numerals designate corresponding elements. The figures are not to scale but are intended to disclose the inventive concepts by illustration. This drawing, together with the description herein, serves to explain the principles of the invention and is only for the purpose of illustrating preferred and alternative examples of how the invention can be made and used.

It will be recognized that the principles of the invention may be utilized and embodied in many and varied forms, and that various materials, component parts and arrangements of components may be employed in utilizing the invention. In order to demonstrate these principles, the invention is described herein by reference to specific preferred embodiments. The invention, however, is not limited to the specific forms illustrated and described in detail.

In the embodiment illustrated the bandage comprises an elongated strip 10 with opposite ends 11, 12 which can be secured to the body of strip 10 with conventional fastening means 20 such as clips, pins, Velcro® fasteners or the like. The elongated strip carries at least one pocket 13 on its inner surface to position a thermal packet 15 such as a gel pak or the like to apply cold (or heat) directly to the affected body part.

In the preferred embodiment, the pocket strip 13 is centrally located along the length of the strip 10 and divided to form adjacent pockets 13a and 13b with the bottoms of the pockets joined and the pocket entries at opposite ends. A hot or cold pak is carried in one or more of the pockets as required to provide the desired hypothermic therapy to the affected body part.

In use, the strip 10 is wrapped around the patient's head as illustrated in FIGS. 5 and 6 so that the pocket 13 is positioned adjacent the affected body part. Compression is applied to the pocket 13 (and thus to the affected body part) by stretching the strip 10 lengthwise before attaching the first end 12 to the inner surface 21 of the strip. The remainder of the strip is then wrapped around the patient's head as illustrated in FIGS. 5 and 6 and the second end 11 secured to the outer surface of the strip 10 to maintain the strip 10 in place and maintain pressure on the body part adjacent the pocket 13. The degree of compression applied is determined by appropriately adjusting the tension applied during such application.

In the preferred embodiment the elongated strip 10 comprises a two-layer stretch bonded laminate of spun-bond polypropylene which is approximately thirty-six (36) to approximately forty-eight (48) inches long and approximately three and one-half (3½) inches wide in the static (non-stretched) state. Fastening means 20 in the form of Velcro® strips are positioned on opposite surfaces of first end 12 and second end 11.

A pocket strip 13 of non-stretchable spun-bond polypropylene is weld-bonded to the inner surface 21. The outer edges of the pocket strip 13 are secured to the outer edges of strip 10 and the longitudinal center is also bonded to the strip 20 to form pockets 13a and 13b with opposite open ends 14a and 14b.

Since the strip 10 is elastic (stretchable) and the pocket strip 13 is non-elastic (non-stretchable), the pocket strip (or pockets 13a, 13b) may be positioned adjacent the affected body part and maintained in position even though the remainder of the strip 10 is stretched to secure the pockets in place and apply compressive force to the body part adjacent the pockets.

In use, a thermal packet 15 is inserted into a pocket (14b as shown in FIG. 3). The thermal packet 15 may be any suitable conventional device such as an ice pak or gel pak commonly used in thermal packet applications. As used herein, “thermal packet” is used to describe any package of material which absorbs thermal energy (such as ice pak or a cold gel pak) or releases thermal energy (such as a heated gel pak).

Since the strip 10 is porous and pocket strip 13 is non-porous, the strip 10 is breathable, permitting passage of air and moisture therethrough. The pocket strip 13, however, is non-porous and prevents the passage of moisture therethrough. Thus the inside surfaces of the pockets 13a, 13b are non-porous while the outside surfaces (defined by strip 10) are porous and breathable. Where ice paks or other thermal packets which collect or release moisture are used, an absorbent material (not shown) may be positioned within the pocket to collect such moisture.

As illustrated in FIG. 3, when a thermal packet 15 is carried in a pocket (such as pocket 13b) the thickness of the packet 15 causes a bulge to extend from the inner surface 21 of the strip 10. When applying the bandage to a human head as illustrated in FIGS. 5 and 6, the inner surface of the pocket 13b is positioned adjacent the affected body part and the first end 12 of strip 10 stretched in one direction (over the head as shown in FIG. 5) and the opposite end 11 drawn in the opposite direction until the inner surface 21 of the strip 10 overlaps the fastener 20 on the outer surface 22 of the strip. The second end 12 is then secured to the inner surface of the strip 10 as shown in FIG. 5. Since the thermal packet 15 bulges inwardly from the surface 21 of the strip 10, stretching the strip 10 around the head urges the pocket 13b against the body part, thus applying compressive force directly to the body part.

Since the strip 10 is elastic and the thermal packet 15 forms a bulge on the inner surface of the strip 10, the compressive force applied can be varied by stretching the strip 10 (thus adjusting the compressive force) before attaching the first end 12 to the inner surface 21 of the strip. The compressive force may then be increased by further stretching the first end 11 to overlap the strip 10 and attaching the first end 11 to the outer surface of the strip 10. In this manner the compressive force can be varied as desired by adjusting the attachment of the first end 11 to the outer surface of the strip 10 without removing the bandage from the patient.

While only exemplary embodiments of the invention have been illustrated and described in detail herein, it will be readily recognized that the principles of the invention may be used in various forms to provide maxillofacial therapeutic treatment and support. It will be appreciated that the invention is not limited to any particular dimensions, materials or arrangement of components. Various materials of construction and conventional components will be found suitable by those skilled in the art, and the arrangement, size and location of the various components may be varied as desired. It is to be understood, therefore, that even though numerous characteristics and advantages of the invention have been set forth in detail herein, the foregoing description, together with details of the structure and function of the various embodiments, is to be considered illustrative only. Various changes and modifications may be made in detail, especially in matters of shape, size and materials as well as arrangement and combination of parts, without departing from the spirit and scope of the invention as defined by the appended claims.

What is claimed:

1. A bandage adapted for maxillofacial application comprising:
a) an elongated elastically expandable strip of wrapping material defining a length, a width, opposite ends, an inner surface and an outer surface;
b) a pocket strip attached to one surface of said elastically expandable strip defining at least one pocket adapted to contain a thermal packet, said pocket strip formed of non-elastic material which prevents passage of moisture therethrough; and
c) closure means adapted to secure said elongated strip in a wrapped position with said pocket adjacent a human body part and apply compressive pressure on said body part.

2. A bandage as defined in claim 1 wherein:
a) said elongated strip comprises an elastic spun-bond polypropylene laminate; and
b) said pocket strip comprises a non-elastic spun-bond polypropylene which is impervious to moisture.

3. A bandage as defined in claim 1 wherein said pocket strip is secured to said elongated strip at the edges of both said strips and a point intermediate the ends of said pocket strip to define two pockets with opposed open ends.

4. A bandage as defined in claim 1 wherein said elongated strip is approximately thirty-six to approximately forty-eight inches long and approximately three and one-half inches wide.

5. A bandage as defined in claim 1 wherein said closure means comprises a Velcro® strip on the inner surface of one end of said elongated strip and a second Velcro® strip on the outer surface of the opposite end of said elongated strip.

6. A method of applying therapeutic thermal compression to a maxillofacial body part of a human head comprising the steps of:
a) forming a bandage comprising:
(i) an elongated elastically expandable strip of wrapping material which defines a width, a length, an inner surface, an outer surface, a first end and a second end;
(ii) a pocket defined by a pocket strip attached to the inner surface of said elongated strip, said pocket having a closed end and an open end; and
(iii) closure means adapted to secure the ends of said strip to a surface of said strip;
b) positioning a thermal packet in said pocket;
c) positioning the pocket containing the thermal packet adjacent a maxillofacial body part on a human head;
d) wrapping the elongated strip around said human head in a position to hold said pocket adjacent said maxillofacial body part while stretching said elongated strip;
e) attaching the first end of said elongated strip to the inner surface of said elongated strip to apply compressive force to said thermal packet; and
f) attaching the second end of said elongated strip to the outer surface of said elongated strip in a position to maintain said elongated strip in a stretched condition and maintain compressive force on said thermal packet in said pocket.

7. The method defined in claim 6 including the step of:
g) adjusting the compressive force applied to said thermal packet by further stretching said second end of said elongated strip before attaching said second end to said elongated strip.
